(19) United States
(12) Patent Application Publication
(10) Pub. No.: US 2018/0250222 A1
(43) Pub. Date: Sep. 6, 2018

(54) SUNSCREEN

(71) Applicant: Continuum Group LLC, Fremont, CA (US)

(72) Inventors: John Lyle, Fremont, CA (US); Victor Fung, Fremont, CA (US)

(21) Appl. No.: 15/907,094

(22) Filed: Feb. 27, 2018

Related U.S. Application Data
(60) Provisional application No. 62/464,259, filed on Feb. 27, 2017.

(51) Int. Cl. A61K 8/99; A61K 8/66; A61Q 17/04

(52) U.S. Cl. CPC A61K 8/99; A61K 2800/75; A61Q 17/04; A61K 8/66

(57) ABSTRACT

As people age, the microbiome of human skin changes through processes of selection due both to external and intrinsic factors. While human lifespan is finite determined by the inevitable decay of human cellular and genomic structures, the lifespan and aging of the microbiome is more ecological in nature, driven by selective pressures both connected and unconnected to human's own aging.

Through the manipulation of the microbiome, using corollaries of processes used in ecosystem restoration, this invention includes ways, compositions and formulations to restore the microbiome of the skin allowing the skin to be in a more youthful and/or native state and prevent or reverse some of the undesirable appearances of aging. The invention described herein restores a putative ancestral microbiome function and thus protects the skin from damage caused by the UV rays present in sunshine.

### CROSS REFERENCE TO RELATED APPLICATIONS

This application claims the benefit under 35 U.S.C. § 119(e) of U.S. Provisional Application No. 62/464,259, filed on Feb. 27, 2017 which is hereby incorporated by reference in its entirety.

### FIELD OF THE INVENTION

The present invention relates to microbe-based compositions and formulations for sunscreen applications.

### BACKGROUND OF THE INVENTION

Exposure to UV rays is the predominant cause of the appearance of aging of skin (Grant W B, Eur J Cancer. 2008, 44(1):12-15). Other factors contributing to the aging of skin include exposure to low humidity, pollution, as well as fatigue, aging, and poor nutrition. UV exposure changes the physical properties, visual appearance, and perhaps physiological functions of skin in ways that result in aesthetically undesirable effects (Flament F, et al., Clin Cosmet Invest Dermatol. 2013, 6:221-232). The most obvious changes are the appearance of lines and wrinkles, changes in the evenness of coloration and tone, reduction in firmness, reduced elasticity resulting in increased sagging, and rough surface texture. Less obvious, there can be a reduction in the ability of the skin to remodel and repair itself, perhaps resulting from the accumulated errors in structure and function, and alterations in the normal regulation of common biochemical pathways due to UV damage to the underlying genetic material (Bosch R, et al., Antioxidants (Basel) 2015, 4(2):248-268).

While use of sunscreen has been demonstrated to slow the appearance of aging, there are many drawbacks to the currently available sunscreens, for example, the unknown effects of nanoparticles in mineral based sunscreen, hormonal effects of sunscreen ingredients, and known carcinogens in the breakdown products of chemical sunscreens (Knowland J, et al., FEBS Letters 1993, 324:309-313; Klann A, et al., Environ. Res. 2005, 97(3):274-281; Krause M, et al., International Journal of Andrology 2012, 35(3):424-436). Therefore, finding and providing a more natural source of protecting the skin from UV rays is highly desirable.

### Overview of the Microbiome

Formation of human microbiome begins at birth. Rapidly, microbial cells outnumber human cells (Sender R, et al., PLoS Biol. 2016, 14(8):e1002533) and the total number of genes in the microbiome outnumbers human genes significantly (Lloyd-Price J, Nature 2017, 550:61-66). Its makeup has been implicated in everything from health to beauty, with the greatest focus on understanding the role of the microbiome in the formation or prevention of disease states. The diversity of disease states with demonstrated dependencies on the microbiome, from those associated with developmental stages such as acne to those considered infections (both skin and gut) as well as autoimmune diseases such as asthma, demonstrates the powerful and highly interactive nature of human's codependence on their microbiome.

While the microbiome is a major determinant of health, it can also contribute to human appearance. There are well known examples of acne and psoriasis, and it has been suggested that the functions of the microbes determine the composition of lipids on the surface of the skin through metabolism of sebum (the oily secretion of human skin). Therefore, the microbiome influences whether or not the skin provides its own moisturizer in those breakdown products, delaying the signs of aging in human skin (Pappas, A, Dematoendocrinol. 2009, 1(2):72-76).

The co-evolution with the microbiome occurred long before the modern standards of cleansing and regular use of antibiotics were adopted. An antimicrobial property is often added to human skin or skin care products including sunscreen via multiple compounds or methods, for example, as described in U.S. Pat. No. 6,613,755; U.S. Pat. No. 6,114,377; U.S. Pat. No. 8,193,244; US Publication No. 20040228811A1 and WO 2012046922. Antibiotics and modern hygiene do protect humans from many pathogens, however, such processes also remove many of the microbes that would be indigenous to human skin. Therefore, selectively reintroducing specific beneficial microbes or restoring desirable process of human ancestral microbiome for UV protection purposes is a preferred path to abandoning all modern practices in sunscreen applications.

### Microbiome as an Ecosystem

Both the sheer numbers of microbial cells and their genomic diversity demonstrate that the microbiome is not a single entity, but rather a complex ecosystem. The human genome may be a dominant force in determining the current state of health or appearance, but it is fixed, at least with the current broadly available technologies.

The microbiome, however, is changeable. This is demonstrated in the negative context by infectious diseases. While there are cases of infectious organisms which can cause a disease state with a single colonization, it is now recognized that disease states can also be the result of the microbiome reaching an equilibrium which does not favor health. Such is the case in the gut microbiome for Clostridium difficile “infection” whose cure, rather than solely antibiotics, is the reintroduction of competitive species (Brandt L J, Gastroenterol Hepatol (N Y) 2012, 8(3):191-194).

Just as disease states may be manipulated or ameliorated by favoring the growth of healthy microorganisms, the present invention includes methods to favor states of the microbiome and/or restore functions of a prior indigenous microbiome which protect against the appearance of aging, favoring a more youthful appearance.

### Skin Microbiome and UV Protection

The skin microbiome is perhaps the most subject to changes in selection pressure through human modern routines. Regular washing, both mechanically and via the antimicrobial activity in soaps, exerts significant selective pressures on this microbial community. The use of antibiotics, both nonspecifically and to target species on the skin as in the case of acne also significantly changes the microbiome of the skin.

The contributors to the appearance of aging have been investigated through twin studies and it has been demonstrated that exposure to the sun is the largest contributor (Guyuron B, et al., Plastic & reconstructive surgery 2009, 123(4):1321-1331). The mechanisms can be diverse, but research implicates UV exposure directly in the loss of the barrier function of the skin, a direct path to loss of moisture, increased exposure of the lower layers to stresses, and increased potential for infection (Biniek K et al., Proc Natl Acad Sci USA 2012, 109(42):17111-17116). Use of sunscreen therefore is a way to protect against the signs of aging and has been widely adopted both to prevent the signs of aging and with a hope to reduce the risk of cancer.

However, despite increased sunscreen use, the incidence rate of skin cancer continues to increase (National Cancer Institute, 2016, FIG. 1). There are several explanations for the disappointing results of increased sunscreen usage. The simplest is that sunscreen is applied ineffectively (Stern R S, N. Engl. J. Med. 2004, 350(15):1526-1534). Another is that photochemical breakdown products of the chemicals in sunscreen are themselves carcinogenic (Knowland J, et al., FEBS Letters 1993, 324:309-313; Hanson K M, et al., Free Radical Biology & Medicine 2006, 41:1205-1212; Mosley C N, et al., Int J. Environ. Res. Public Health 2007, 4(2):126-131; Gonzalez H, et al., BMC Dermatol. 2007, 7(1):1-9.). Another is that the lack of vitamin D due to the lack of UV exposure is a cause for the continuing increase in incidence rate. While it is clear that sunburn and intermittent UV exposure are risk factors for skin cancer, continuous exposure to the sun actually results in reduced risk (Gandini S, et al., European Journal of Cancer 2005, 41(1):45-60). This is further backed up by the distribution of skin cancer cases with latitude in the United States, with rates higher in northern states where people presumably have more intermittent exposure (National Cancer Institute, 2016, FIG. 2).

### BRIEF SUMMARY OF THE INVENTION

The present invention provides topical compositions and formulations for sunscreen applications. It is directed to microbe-based compositions and their products for maintaining or restoring the appearance of the skin in or to a youthful phenotype.

In one embodiment, a topical formulation is provided comprising one or more strains of microbes under appropriate conditions for continued bacterial viability and for application to the skin of a subject.

In another embodiment, the topical formulation is used for maintaining the appearance of the skin in a youthful phenotype or restoring the appearance of the skin to a youthful phenotype.

In another embodiment, the microbes excrete UV absorbing materials.

In another embodiment, the microbes excrete molecules that prevent or reduce photochemical damage caused by UV radiation.

In one exemplary embodiment, the microbes are selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp.

In another exemplary embodiment, the microbes are from the genus of Agrococcus, Bacillus, Brevibacterium, Curtobacterium, Kocuria, Microbacterium, Micrococcus, Paenibacillus, Paracoccus, Phycibacter, Pseudomonas, Staphylococcus, or Terribacillus.

In another exemplary embodiment, the microbes are selected from the group consisting of Agrococcus terreus, Bacillus sp. BAB-4122, Bacillus anthraces, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus.

A topical formulation is also provided according to one embodiment, comprising products of bacterial growth for application to the skin of a subject, while the products of bacterial growth are selected from the group consisting of enzymes, metabolic by-products and peptides.

In another embodiment, the aforementioned formulation is used for maintaining the appearance of the skin in a youthful phenotype or restoring the appearance of the skin to a youthful phenotype.

In another embodiment, the products of bacterial growth comprise UV absorbing materials.

In another embodiment, the products of bacterial growth prevent or reduce photochemical damage caused by UV radiation.

In one exemplary embodiment, the products of bacterial growth are from microbes selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp.

In another exemplary embodiment, the products of bacterial growth are from microbes which are from the genus of Agrococcus, Bacillus, Brevibacterium, Curtobacterium, Kocuria, Microbacterium, Micrococcus, Paenibacillus, Paracoccus, Phycibacter, Pseudomonas, Staphylococcus, or Terribacillus.

In another exemplary embodiment, the products of bacterial growth are from microbes selected from the group consisting of Agrococcus terreus, Bacillus sp. BAB-4122, Bacillus anthraces, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus.

A topical formulation is also provided according to one embodiment, comprising a combination of one or more strains of microbes under appropriate conditions for continued bacterial viability and products of bacterial growth for application to the skin, wherein the products of bacterial growth are selected from the group consisting of enzymes, metabolic by-products and peptides.

In another embodiment, the aforementioned formulation is used for maintaining the appearance of the skin in a youthful phenotype or restoring the appearance of the skin to a youthful phenotype.

In another embodiment, the microbes excrete UV absorbing materials and the products of bacterial growth comprise UV absorbing materials.

In another embodiment, the microbes excrete UV absorbing materials or the products of bacterial growth comprise UV absorbing materials.

In another embodiment, the microbes produce materials that prevent or reduce photochemical damage caused by UV radiation and the products of bacterial growth prevent or reduce photochemical damage caused by UV radiation.

In another embodiment, the microbes produce materials that prevent or reduce photochemical damage caused by UV radiation or the products of bacterial growth prevent or reduce photochemical damage caused by UV radiation.

In one exemplary embodiment, the microbes are selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp.; and/or the products of bacterial growth are from microbes selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp.

In another exemplary embodiment, the microbes are from the genus of Agrococcus, Bacillus, Brevibacterium, Curtobacterium, Kocuria, Microbacterium, Micrococcus, Paenibacillus, Paracoccus, Phycibacter, Pseudomonas, Staphylococcus, and/or Terribacillus.; and/or the products of bacterial growth are from microbes which are from the genus of Agrococcus, Bacillus, Brevibacterium, Curtobacterium, Kocuria, Microbacterium, Micrococcus, Paenibacillus, Paracoccus, Phycibacter, Pseudomonas, Staphylococcus, and/or Terribacillus.

In another exemplary embodiment, the microbes are selected from the group consisting of Agrococcus terreus, Bacillus sp. BAB-4122, Bacillus anthraces, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus; and/or the products of bacterial growth are from microbes selected from the group consisting of Agrococcus terreus, Bacillus sp. BAB-4122, Bacillus anthraces, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus.

In one embodiment, the topical formulation does not comprise a synthetic small molecule UV absorption agent.

In another embodiment, the topical formulation is used for application to one or more regions of the skin, while the one or more regions of the skin do not comprise microbes excreting UV absorbing materials.

In another embodiment, the topical formulation does not cause substantially skin irritation after being applied to the skin.

In still another embodiment, the topical formulation does not comprise a synthetic small molecule UV absorption agent; and the topical formulation is used for application to one or more regions of the skin, while the one or more regions do not comprise microbes excreting UV absorbing materials.

In still another embodiment, the topical formulation does not comprise a synthetic small molecule UV absorption agent; and the topical formulation does not cause substantially skin irritation after being applied to the skin.

In still another embodiment, the topical formulation is used for application to one or more regions of the skin, while the one or more regions do not comprise microbes excreting UV absorbing materials; and the topical formulation does not cause substantially skin irritation after being applied to the skin.

In still another embodiment, the topical formulation does not comprise a synthetic small molecule UV absorption agent and is used for application to one or more regions of the skin where the one or more regions do not comprise microbes excreting UV absorbing materials; and the topical formulation does not cause substantially skin irritation after being applied to the skin.

In still another embodiment, the topical formulation of the present invention does not comprise an antimicrobial agent.

These and other embodiments, features and potential advantages will become apparent with reference to the following description and drawings.

### BRIEF DESCRIPTION OF THE DRAWINGS

FIG. 1: Increasing incidence of skin cancer. Trends in incidence rates, 1975-2013, from the National Cancer Institute, 2016. Despite a large increase in sunscreen use, incidences of skin cancer continue to rise.

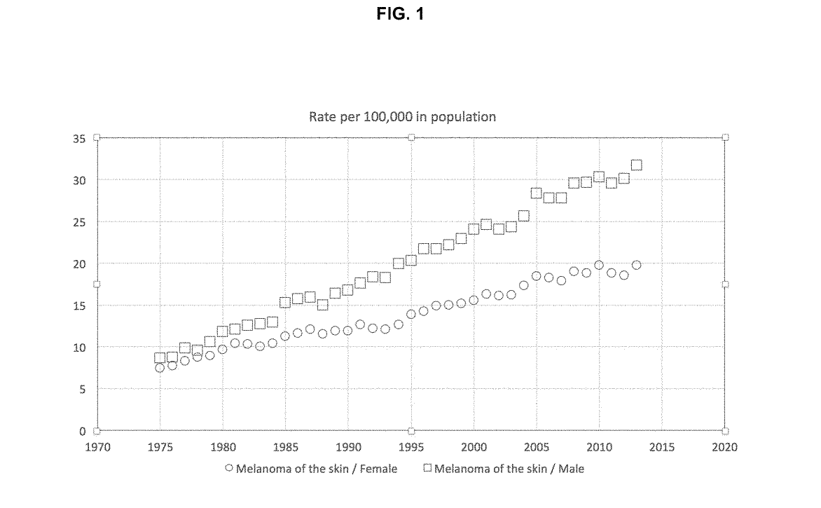

FIG. 2: Skin cancer rates by state. Rates are higher in northern states where exposure to UV rays is assumed to be less.

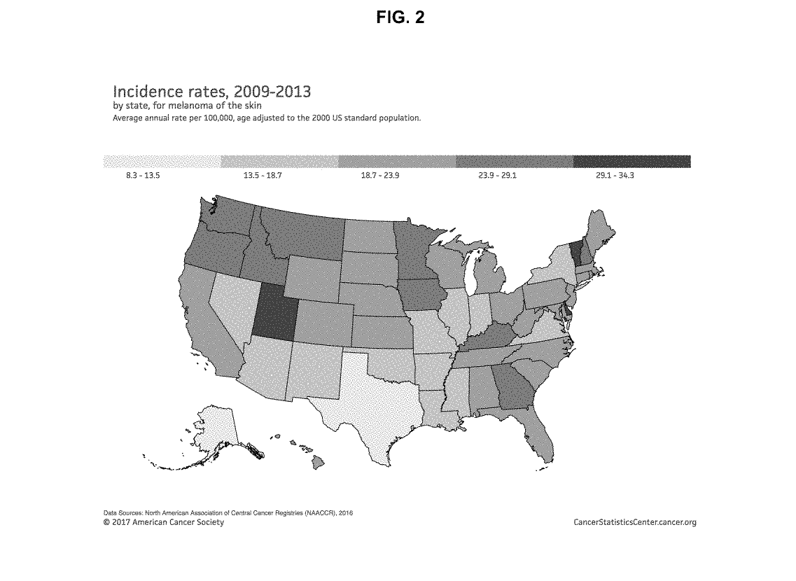

FIG. 3: Microbes isolated from the surface of the skin of individuals with significant sun exposure who do not use sunscreen demonstrate resistance to UV-B radiation. Microbes were spread horizontally across the plate. The right hand side of the plate was then exposed to 302 nm UV radiation for 15 minutes. The plate was then incubated in the dark and photographed the following day. Clear growth in the UV exposed section was observed for UV resistant strains, designated 3E4 (SS20), JG8W (SS49), and JG13 (SS50) (one Microbacterium ssp. and two Terribacillus ssp.), while no growth was observed for UV sensitive strains, designated 3E1, 3E2, and 3E3.

FIG. 4: Time course of UV absorption curves of culture media from a UV resistant microbe demonstrating secretion of UV absorbing materials. LB media was inoculated with a UV resistant microbe (a Paenibacillus ssp.). The culture was incubated at 37° C. in a shaking incubator. Samples of culture were removed, microbes were pelleted by centrifugation, and absorbance of the clarified media was measured in a UV-Vis spectrophotometer at the noted times. The diamond denoted curve is the baseline (immediately post inoculation of the culture). The square, triangle, and circle denoted curves are 2, 5, and 8 days post inoculation respectively.

FIG. 5: Reduced photochemistry with cultures of UV absorbing microbes. An indicator dye was mixed with a variety of UV resistant cultures and the mixture was then exposed to UV radiation at 302 nm for 24 hours. After centrifugation to clarify the solution of microbes, the absorbance at 560 nM was measured using a spectrophotometer. A decrease in absorption (Y axis) is seen with photochemistry due to UV exposure in the absence of products from a culture of UV resistant microbes.

FIG. 6: Effective spectrum of UV radiation on human skin (McKinlay A F, et al., CIE Journal 1987, 6(1):17-22.). The derivation of the effective spectrum of UV radiation, or the proportional potential of UV radiation to cause damage to the human skin by wavelength is shown. This effective spectrum is the convolution of the ability of a particular wavelength to damage the skin and the prevalence of a particular wavelength in the sunlight which reaches the surface of the earth. A perfectly efficient sunscreen would mimic this curve in its absorbance.

FIG. 7A: UV Absorbance of strains with 16S homology to Micrococcus luteus (SS21), Micrococcus yunnanensis (SS25 & SS26), Staphylococcus saprophyticus (SS41 & SS42). As in FIG. 4, LB media was inoculated with a microbe isolated from an individual that has significant sun exposure and does not use sunscreen (both self-reported). After 5 days of growth, culture media was removed, microbes were pelleted by centrifugation, and absorbance of the clarified media was measured in a UV-Vis spectrophotometer. Absorbance has been normalized to a maximum absorbance equal to one. The effective spectrum is shown for reference. Strains presented secrete materials into the media which have significant absorbance across the effective spectrum, either as absolute absorbance or when normalized against the effective spectrum.

FIG. 7B: UV Absorbance of strains with 16S homology to a Micrococcus sp. (SS22), Micrococcus yunnanensis (SS27), Staphylococcus saprophyticus (SS43 & SS44). See legend to FIG. 7A for a full description.

FIG. 7C: UV Absorbance of strains with 16S homology to Curtobacterium citreum (SS16), Staphylococcus arlettae (SS31), Staphylococcus haemolyticus (SS36), Terribacillus saccharophilus (SS48), Bacillus thurigiensis and Bacillus cereus (SS51). See legend to FIG. 7A for a full description.

FIG. 7D: UV Absorbance of strains with 16S homology to Bacillus anthraces and Bacillus cereus (SS3), Bacillus firmus (SS6) Bacillus sp. BAB-4122 (SS12), Staphylococcus capitis and Staphylococcus caprae (SS32), Staphylococcus haemolyticus (SS37 & SS38). See legend to FIG. 7A for a full description.

FIG. 7E: UV Absorbance of strains with 16S homology to Bacillus oleronius (SS7), Staphylococcus epidermidis (SS33 & SS34), Staphylococcus nepalensis (SS39 & SS40), Staphylococcus saprophyticus and Staphylococcus xylosus (SS45). See legend to FIG. 7A for a full description.

FIG. 7F: UV Absorbance of strains with 16S homology to Bacillus simplex and Brevibacterium frigoritolerans (SS8), Paracoccus acridae (SS30) a Staphylococcus sp. (SS47), Staphylococcus epidermidis (SS52). See legend to FIG. 7A for a full description.

FIG. 7G: UV Absorbance of strains with 16S homology to Agrococcus terreus (SS2), Bacillus aquimaris and Bacillus marisflavi (SS5), a Bacillus sp. (SS10), Kocuria turfanensis (SS17), Microbacterium lacticum and Microbacterium foliorum (SS18), a Micrococcus sp. (SS23 & SS24). See legend to FIG. 7A for a full description.

FIG. 7H: UV Absorbance of strains with 16S homology to Brevibacterium pityocampae (SS14 & SS15), Micrococcus yunnanensis (SS28), a Staphylococcus sp. (SS46). See legend to FIG. 7A for a full description.

FIG. 7I: UV Absorbance of strains with 16S homology to Bacillus aquimaris (SS4), a Bacillus sp. (SS11), a Paenibacillus sp. (SS29), Staphylococcus epidermidis (SS35). See legend to FIG. 7A for a full description.

FIG. 7J: UV Absorbance of strains with 16S homology to Bacillus simplex and Brevibacterium frigoritolerans (SS9), a Bacillus ssp. (SS13), a Terribacillus sp. (SS49 & SS50). See legend to FIG. 7A for a full description.

FIG. 7K: UV Absorbance of strains with 16S homology to Pseudomonas fulva (SS53) and Pseudomonas putida (SS54). See legend to FIG. 7A for a full description.

FIG. 8: Skin safety testing pilot. Two candidate strains (a Staphylococcus sciuri strain and a Bacillus sp.) were swabbed onto the right forearm of the test subject. An initial image (left) was captured immediately after the swab. A second image (center) was taken after 30 minutes. Notable irritation to and reddening of the skin occurred with the Staphylococcus sciuri strain at this time point. This strain was omitted from further consideration and removed from the surface of the skin. A third image (right) was taken after 10 hours. No irritation was noted for the Bacillus sp. Continued observation for 1 week showed no notable irritation or changes to the surface of the skin with this strain (data not shown).

### DETAILED DESCRIPTION OF THE INVENTION

### Introduction

The modern intermittent exposure to the sun, along with the use of conventional sunscreen removes the selective advantage given to commensal microbes on human skin that produce their own sunscreen that would also provide effective sunscreen to the human skin, their host. This along with modern washing and antibiotic use has resulted in the removal of the indigenous microbial sunscreen and therefore exposes people to UV rays to a higher degree whenever they fail to use sunscreen. Compounding the loss, unlike the synthetic, aromatic hydrocarbons or nanoparticles that make up commercial sunscreen, the sunscreen molecules secreted by human ancestral microbiome and their photochemical products would be less likely to be carcinogenic as these effects would have been detrimental to both the microbe and the host and thus would have been selected against.

It was showed in the Applicants' study that individuals exposed to sun on regular occasion who don't use sunscreen have microbes colonizing the surface of their skin that secrete UV absorbing materials, providing UV protection to both the microbe and the host. Microbes secrete their sunscreen molecules rather than producing them internally as their size precludes an effective sunscreen given the space available within the microbial cell. Multiple strains with UV resistance have been isolated from individuals with regular exposure to the sun. However, no such microbes were identified from the faces of individuals with infrequent exposure to the sun.

While the existence of naturally produced sunscreens has been extensively studied in aquatic organisms, there is a lack of study of sunscreen molecules produced by microbes colonizing humans. The invention described herein restores the function of the earlier microbiome in providing UV protection to those who lack it by restoring protective bacteria, the secreted sunscreen of those bacteria, or a combination of those.

### Definitions

Unless otherwise defined herein, scientific and technical terms used in connection with the present invention shall have the meanings that are commonly understood by those of ordinary skill in the art. The meaning and scope of the terms should be clear. However, in the event of any latent ambiguity, definitions provided herein take precedence over any dictionary or extrinsic definition. Further, unless otherwise required by context, singular terms shall include pluralities and plural terms shall include the singular. Furthermore, the use of the term “including,” “includes,” or “included” is not limiting. Terms such as “element” and “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, delivery, and treatment of patients.

All headings and section designations are used for clarity and reference purposes only and are not to be considered limiting in any way. For example, those of skill in the art will appreciate the usefulness of combining various aspects of the disclosure from different headings and sections as appropriate according to the spirit and scope of the invention described herein.

Select terms are defined below in order for the present invention to be more readily understood.

The term “skin” refers to one or more of the epidermis, dermis, and hypodermis (i.e., subcutis), hair follicles, hair roots, hair bulbs, the ventral epithelial layer of the nail bed (lectulus), sebaceous glands, eccrine and apocrine sweat glands.

The term “topical” and variations thereof refer to compositions that are intended to be applied directly to the outer surface of the skin or other keratinous tissue. The topical application of the compositions of the invention reduces damages to the skin which are caused by exposure to sunlight or to other sources of UV radiations.

The term “topical composition” refers to a composition suitable for application to mammalian, e.g., human, skin. Non-limiting examples of topical compositions include skin care formulations such as cleansers, toners, serums, sticks, wipes, masks, lotions, creams, ointments, balms, oils, scrubs, liquid, bar, gel, oil, foam or treatments; as well as cosmetic products, including, but not limited to, foundations, eye liners, eye shadows, blushes, bronzers, highlighters, lip liners, brow pencils, blemish/beauty balm creams, color correcting/control creams, lipsticks, mascaras, lip glosses, lip balms, concealers, and powders. Topical compositions optionally regulate and/or improve various skin conditions or provide a desired visual effect. For example, a topical composition can provide one or more of the following effects: improve skin appearance and/or texture; increase the thickness of one or more layers of the skin; improve the elasticity or resiliency of the skin, improve the firmness of the skin; reduce the oily, shiny, or dull appearance of skin; improve the hydration status or moisture content of skin; minimize the appearance of fine lines or wrinkles; improve skin exfoliation or desquamation; soften the skin; improve skin barrier function; improve skin tone; reduce inflammation, redness or skin blotches or hyperpigmentation; and improve the brightness, radiance, or translucency of skin.

The term “topical formulation” as used herein refers to a formulation intended for topical or local application to a region of a subject in need thereof, and includes but not limited to formulation forms such as gel, cream, ointment, emulsion, lotion, stick, serum, wipe, spray, film, liquid, bar or foam.

The term “microbe” and “microorganism” are synonymous and refer to bacteria, fungi, and algae.

The term “cream” refers to a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes.

The term “ointment” refers to a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

The term “gel” refers to a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

The term “lotion” refers to a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion in the form of an emulsion having a viscosity of between 100 and 1000 centistokes. The fluidity of lotions permits rapid and uniform application over a wide surface area. Lotions are typically intended to dry on the skin leaving a thin coat of their functional components on the skin's surface.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

The term “maintaining the appearance of the skin in a youthful phenotype” as used herein refers to maintaining a perceptible positive benefit in the appearance, texture, and/or function of skin. The term “restoring the appearance of the skin to a youthful phenotype” as used herein refers to effecting a perceptible positive change in the appearance, texture, and/or function of skin. A perceptible positive benefit or change can include, for example, one or more of the following: reducing the appearance of wrinkles, coarse deep lines, fine lines, crevices, bumps and/or large pores; increasing the collagen and/or elastin content of the skin; lightening the skin; increasing skin elasticity; maintaining or improving skin barrier function; preventing or reducing skin redness; preventing or reducing inflammation; improving the coloration of the skin, for example, reducing under-eye circles, sallowness, dullness, and hyperpigmentation; and increasing moisture content of the skin.

The term “signs of aging” includes but not limited to the following signs: the appearance of wrinkles, coarse deep lines, fine lines, crevices and/or large pores; a decrease in collagen and/or elastin content of the skin; a decrease in skin elasticity and/or skin barrier function; darkening of the skin; a decrease in the moisture content of the skin; and/or appearance of under-eye circles, sallowness, dullness, and hyperpigmentation.

The term “photochemical damage caused by UV radiation” refers to any adverse effects caused by the absorption of UV light by a molecular structure, such as proteins, nucleic acids (e.g. DNA, RNA), chemicals etc. Many proteins and nucleic acids absorb UV light and are denatured or damaged by the radiation. The term “photochemical damage caused by UV radiation” also includes damage(s) caused by photochemical breakdown products of the chemicals in a sunscreen (e.g. the active ingredients in a sunscreen). Photochemical damage caused by UV radiation in this invention includes but not limited to erythema, sunburn, skin rash, skin pigment formation or darkening and development of skin cancer.

The term “small molecule” refers to a low molecular weight (<900 daltons) compound with a size on the order of 1 nm.

The term “skin irritation” refers to skin reaction to a particular irritant that results in for example, inflammation of the skin, skin rash and/or skin itchiness.

### Topical Compositions

The composition described herein is comprised of one or more strains of microbes under appropriate conditions for continued bacterial viability and/or products of microbial growth for topical application to the skin of a subject.

In one exemplary embodiment, the microbes are selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp.

In another exemplary embodiment, the microbes are from the genus of Agrococcus, Bacillus, Brevibacterium, Curtobacterium, Kocuria, Microbacterium, Micrococcus, Paenibacillus, Paracoccus, Phycibacter, Pseudomonas, Staphylococcus, and/or Terribacillus.

In another exemplary embodiment, the microbes are selected from the group consisting of Agrococcus terreus, Bacillus sp. BAB-4122, Bacillus anthraces, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus.

The composition described herein comprises any desired amount of fermented media, microbial culture, purified bacteria or combination thereof. The amounts of these components are individually or in combination about 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, or more (or any range or integer therein), by weight or volume of the extract or combination of extracts. In one preferred embodiment, the amount of the fermented media described above is 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%. In another preferred embodiment, the amounts of purified compounds from the media described above are individually or in combination 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20%. In another preferred embodiment, the amount of purified bacteria described above is equal to or more than 1000 colony forming units (CFU) per dose.

The compositions of the present invention described herein have a pH between about 4.5 to about 9. Depending on the type of composition desired, the pH is 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5 or 14 or any integer or derivative therein. In one embodiment, the pH is 4, 4.5, 5, 5.5, 6, 6.5 or 7. In another embodiment, the pH is 4, 4.5, 5 or 5.5. In still another embodiment, the pH is 4. In still another embodiment, the pH is 4.5. In still another embodiment, the pH is 5. In still another embodiment, the pH is 5.5.

In an exemplary embodiment, the compositions of the present invention comprise a triglyceride, and the non-limiting examples are small, medium, and large chain triglycerides. In a preferred embodiment, the compositions of the present invention comprise a medium chain triglyceride (e.g., caprylic capric triglyceride).

In one embodiment, the compositions of the present invention comprise additional ingredients usually used in the fields under consideration, and/or include dyestuffs that are soluble in the medium of the composition, solid particles such as fillers or pigments, antioxidants, preserving agents, fragrances, electrolytes, solvents, aqueous-phase or oily-phase thickeners or gelling agents, neutralizers, film-forming polymers, cosmetic and pharmaceutical active agents with a beneficial effect on the skin or keratin fibers, and mixtures thereof. These additives present in the composition in the amounts generally used in cosmetics and dermatology. In one embodiment, these additives constitute a proportion of from 0.01% to 90%, or any integer or derivative therein relative to the total weight of the composition. In one embodiment, the amount of the additives described above is from 0.1% to 10% of the total weight of the composition. In one preferred embodiment, the amount of the additives described above in the media is from 50% to 90% of the total weight of the composition. In another preferred embodiment, the amount of the purified additives from the media is from 80% to 99.9% of the total weight of the composition. In one exemplary embodiment, these additives are introduced into the aqueous phase, the fatty phase, and/or into the lipid vesicles and especially liposomes.

One skilled in the art can adjust the viscosity of the composition of the present invention to achieve a desired result. In one exemplary embodiment, the viscosities of the compositions range from about 1 centipoise (cps) to over 1 million cps or any range or integer derivable therein (e.g., 2 cps, 3 cps, 4 cps, 5 cps, 6 cps, 7 cps, 8 cps, 9 cps, 10 cps, 15 cps, 20 cps, 25 cps, 30 cps, 35 cps, 40 cps, 45 cps, 50 cps, 55 cps, 60 cps, 65 cps, 70 cps, 75 cps, 80 cps, 85 cps, 90 cps, 95 cps, 100 cps, 150 cps, 200 cps, 250 cps, 300 cps, 350 cps, 400 cps, 450 cps, 500 cps, 550 cps, 600 cps, 650 cps, 700 cps, 750 cps, 800 cps, 850 cps, 900 cps, 950 cps, 1000 cps, 2000 cps, 3000 cps, 4000 cps, 5000 cps, 6000 cps, 7000 cps, 8000 cps, 9000 cps, 10000 cps, 20000 cps, 30000 cps, 40000 cps, 50000 cps, 60000 cps, 70000 cps, 80000 cps, 90000 cps, 100000 cps, 200000 cps, 300000 cps, 400000 cps, 500000 cps, 600000 cps, 700000 cps, 800000 cps, 900000 cps, 1000000 cps, etc., as measured on a Brookfield Viscometer using a TC spindle at 2.5 rpm at 25 degrees Celsius). In a preferred embodiment, the viscosities of the compositions described above range from 15,000 cps to 150,000 cps. In another preferred embodiment, the viscosity of the composition described above is 15,000 cps, 20,000 cps, 25,000 cps, 30,000 cps, 35,000 cps, 40,000 cps, 45,000 cps, 50,000 cps, 55,000 cps, 60,000 cps, 65,000 cps, 70,000 cps, 75,000 cps, 80,000 cps, 85,000 cps, 90,000 cps, 95,000 cps, 100,000 cps, 105,000 cps, 110,000 cps, 115,000 cps, 120,000 cps, 125,000 cps, 130,000 cps, 135,000 cps, 140,000 cps, 145,000 cps or 150,000 cps.

In some embodiments, the composition described herein includes one or more excipients to provide the desired form and a desired viscosity, flow or other physical or chemical characteristics for effective application, coverage and adhesion to the skin.

In one embodiment, a topical formulation of this invention comprises a species or multiple species of microbes in a suitable form for continued bacterial viability and for application to the skin for the purpose of maintaining or restoring the appearance of the skin in or to a youthful phenotype.

In another embodiment, the topical formulation described above in which the microbes that excrete UV absorbing materials are used. In one exemplary embodiment, the UV absorbing materials described above comprise mycosporine-like amino acids, scytonemin and/or melanin-derived compounds.

In one exemplary embodiment, the microbes are Agrococcus sp., Bacillus sp., Corynebacterium sp., Microbacteriaceae sp., Micrococcus sp., Microbacterium sp., Phycibacter sp., Staphylococcus sp., Terribacillus sp., and/or Micrococcus sp.

In another exemplary embodiment, the microbes are from the genus of Agrococcus, Bacillus, Corynebacterium, Microbacteriaceae, Micrococcus, Microbacterium, Phycibacter, Staphylococcus, Terribacillus, and/or Micrococcus. In another exemplary embodiment, the microbes are Bacillus simplex, Bacillus aquimaris, Bacillus marisflavi, Staphylococcus ludgensis, Terribacillus saccharophilus, Terribacillus aidingensis, and/or Terribacillus goriensus.

In still another embodiment, the microbes excrete retinol, retinol derivatives, or other active compounds to reduce the signs of aging.

In still another embodiment, the microbes excrete moisturizing molecules.

In still another embodiment, the microbes excrete molecules that prevent or slow photochemistry.

In one embodiment, a topical formulation of this invention comprises the products of bacterial growth (including but not limited to enzymes, metabolic by-products and peptides) for application to the skin for the purpose of maintaining or restoring the appearance of the skin in or to a youthful phenotype.

In another embodiment, the products of bacterial growth contain UV absorbing materials. In one exemplary embodiment, the UV absorbing materials described above comprise mycosporine-like amino acids, scytonemin and/or melanin-derived compounds.

In one exemplary embodiment, the products of bacterial growth are from microbes of Agrococcus sp., Bacillus sp., Corynebacterium sp., Microbacteriaceae sp., Micrococcus sp., Microbacterium sp., Phycibacter sp., Staphylococcus sp., Terribacillus sp., and/or Micrococcus sp.

In another exemplary embodiment, the products of bacterial growth are from microbes in the genus of Agrococcus, Bacillus, Corynebacterium, Microbacteriaceae, Micrococcus, Microbacterium, Phycibacter, Staphylococcus, Terribacillus, and/or Micrococcus. In another exemplary embodiment, the products of bacterial growth are from microbes selected from Bacillus simplex, Bacillus aquimaris, Bacillus marisflavi, Staphylococcus ludgensis, Terribacillus saccharophilus, Terribacillus aidingensis, and/or Terribacillus goriensus.

In still another embodiment, the products of bacterial growth contain retinol, retinol derivatives, or other active compounds which reduce the signs of aging.

In still another embodiment, the products of bacterial growth contain moisturizing molecules.

In still another embodiment, the products of bacterial growth contain materials that prevent or slow photochemistry.

In one embodiment, a topical formulation of this invention comprises a combination of a species or multiples species of microbes in a suitable form for continued bacterial viability and the products of bacterial growth (including but not limited to enzymes, metabolic by-products and peptides) for application to the skin for the purpose of maintaining or restoring the appearance of the skin in or to a youthful phenotype.

In another embodiment, the microbes produce and/or the products of bacterial growth contain UV absorbing materials. In one exemplary embodiment, the UV absorbing materials described above comprise mycosporine-like amino acids, scytonemin and/or melanin-derived compounds.

In one exemplary embodiment, the microbes are and/or the products of bacterial growth are from microbes of Agrococcus sp., Bacillus sp., Corynebacterium sp., Microbacteriaceae sp., Micrococcus sp., Microbacterium sp., Phycibacter sp., Staphylococcus sp., Terribacillus sp., and/or Micrococcus sp.

In another exemplary embodiment, the microbes are and/or the products of bacterial growth are from microbes in the genus of Agrococcus, Bacillus, Corynebacterium, Microbacteriaceae, Micrococcus, Microbacterium, Phycibacter, Staphylococcus, Terribacillus, and/or Micrococcus. In another exemplary embodiment, the microbes are and/or the products of bacterial growth are from microbes of Bacillus simplex, Bacillus aquimaris, Bacillus marisflavi, Staphylococcus ludgensis, Terribacillus saccharophilus, Terribacillus aidingensis, and/or Terribacillus goriensus.

In still another embodiment, the microbes produce and/or the products of bacterial growth contain retinol, retinol derivatives, or other active compounds which reduce the signs of aging.

In still another embodiment, the microbes produce and/or the products of bacterial growth contain moisturizing molecules.

In still another embodiment, the microbes produce and/or the products of bacterial growth contain materials that prevent or slow photochemistry.

In still another embodiment, the topical formulation of the present invention does not comprise an antimicrobial agent.

### Restoration of UV Protection Materials to the Skin

Prior to inclusion of the composition described herein in a formulation, microbes are screened for pathogenicity and/or the potential for pathogenesis, ruling out known pathogens and looking for mobile elements in the genome that constitute the other likely means of pathogenesis (Keen E C, Frontiers in cellular and infection microbiology 2012, 2 (article 161):1-3).

Formulations are selected both for their desired properties as a topical product and for their compatibility with the micro-organisms to be restored to the skin via the commonly known technique, such as colony counting assays.

Formulations containing the microbes whose growth products are UV protective materials, the growth products themselves, or a combination thereof are applied directly to the skin.

In a preferred embodiment, the compositions or formulations of the present invention allows the survival of a fraction of some or all of the microbes present for the shelf-life of the material.

In one embodiment, a topical formulation is provided comprising one or more strains of microbes under appropriate conditions for continued bacterial viability and for application to the skin of a subject.

In another embodiment, the topical formulation is used for maintaining the appearance of the skin in a youthful phenotype or restoring the appearance of the skin to a youthful phenotype.

In another embodiment, the microbes excrete UV absorbing materials.

In another embodiment, the microbes excrete molecules that prevent or reduce photochemical damage caused by UV radiation.

A topical formulation is also provided according to one embodiment, comprising products of bacterial growth for application to the skin of a subject, while the products of bacterial growth are selected from the group consisting of enzymes, metabolic by-products and peptides.

In another embodiment, the aforementioned formulation is used for maintaining the appearance of the skin in a youthful phenotype or restoring the appearance of the skin to a youthful phenotype.

In another embodiment, the products of bacterial growth comprise UV absorbing materials.

In another embodiment, the products of bacterial growth prevent or reduce photochemical damage caused by UV radiation.

In one exemplary embodiment, the products of bacterial growth are from microbes selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp.

In another exemplary embodiment, the products of bacterial growth are from microbes which are from the genus of Agrococcus, Bacillus, Brevibacterium, Curtobacterium, Kocuria, Microbacterium, Micrococcus, Paenibacillus, Paracoccus, Phycibacter, Pseudomonas, Staphylococcus, and/or Terribacillus.

In another exemplary embodiment, the products of bacterial growth are from microbes selected from the group consisting of Agrococcus terreus, Bacillus sp. BAB-4122, Bacillus anthraces, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and/or Terribacillus goriensus.

A topical formulation is also provided according to one embodiment, comprising a combination of one or more strains of microbes under appropriate conditions for continued bacterial viability and products of bacterial growth for application to the skin, wherein the products of bacterial growth are selected from the group consisting of enzymes, metabolic by-products and peptides.

In another embodiment, the aforementioned formulation is used for maintaining the appearance of the skin in a youthful phenotype or restoring the appearance of the skin to a youthful phenotype.

In another embodiment, the microbes excrete UV absorbing materials and the products of bacterial growth comprise UV absorbing materials.

In another embodiment, the microbes excrete UV absorbing materials or the products of bacterial growth comprise UV absorbing materials.

In another embodiment, the microbes produce materials that prevent or reduce photochemical damage caused by UV radiation and the products of bacterial growth prevent or reduce photochemical damage caused by UV radiation.

In another embodiment, the microbes produce materials that prevent or reduce photochemical damage caused by UV radiation or the products of bacterial growth prevent or reduce photochemical damage caused by UV radiation.

In one exemplary embodiment, the microbes are selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp; and/or the products of bacterial growth are from microbes selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp.

In another exemplary embodiment, the microbes are from the genus of Agrococcus, Bacillus, Brevibacterium, Curtobacterium, Kocuria, Microbacterium, Micrococcus, Paenibacillus, Paracoccus, Phycibacter, Pseudomonas, Staphylococcus, and/or Terribacillus; and/or the products of bacterial growth are from microbes which are from the genus of Agrococcus, Bacillus, Brevibacterium, Curtobacterium, Kocuria, Microbacterium, Micrococcus, Paenibacillus, Paracoccus, Phycibacter, Pseudomonas, Staphylococcus, and/or Terribacillus.

In another exemplary embodiment, the microbes are selected from the group consisting of Agrococcus terreus, Bacillus sp. BAB-4122, Bacillus anthracis, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus; and/or the products of bacterial growth are from microbes selected from the group consisting of Agrococcus terreus, Bacillus sp. BAB-4122, Bacillus anthracis, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus.

In one embodiment, the compositions or formulations of the present invention have UVA and/or UVB absorption properties. UV absorption agents that are used in combination with the compositions of the present invention include but not limited to physical and chemical sunblocks. In one exemplary embodiment, the physical sunblocks are petrolatum and metal oxides (e.g., titanium dioxide and zinc oxide), kaolin, and talc. In another exemplary embodiment, the chemical sunblocks that are used include para-aminobenzoic acid (PABA), ethyl PABA, ethyl dihydroxypropyl PABA, PABA esters (glyceryl PABA, amyldimethyl PABA and octyldimethyl PABA), butyl PABA, benzophenones (oxybenzone, sullisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octyocrylene, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hyrdoxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and/or isopentyl 4-methoxycinnamate.

In another embodiment, the compositions or formulations of the present invention does not comprise a synthetic small molecule UV absorption agent.

In one exemplary embodiment, the compositions or formulations of the present invention have a sun protection factor (SPF) of 2, 3, 4, 5, 6, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150 or more, or any integer or derivative therein. In a preferred embodiment, the compositions or formulations of the present invention have a SPF of 10, 11, 12, 13, 14, 15, 16, 17, 18, 19, 20, 21, 22, 23, 24, 25, 26, 27, 28, 29 or 30.

In one embodiment of the present invention, the compositions are storage stable. In another embodiment of the present invention, the compositions are color stable. In still another embodiment of the present invention, the compositions are both storage stable and color stable.

In one embodiment, the topical formulation does not comprise a synthetic small molecule UV absorption agent.

In another embodiment, the topical formulation is used for application to one or more regions of the skin, while the one or more regions of the skin do not comprise microbes excreting UV absorbing materials.

In another embodiment, the topical formulation does not cause substantially skin irritation after being applied to the skin.

In still another embodiment, the topical formulation does not comprise a synthetic small molecule UV absorption agent; and the topical formulation is used for application to one or more regions of the skin, while the one or more regions do not comprise microbes excreting UV absorbing materials.

In still another embodiment, the topical formulation does not comprise a synthetic small molecule UV absorption agent; and the topical formulation does not cause substantially skin irritation after being applied to the skin.

In still another embodiment, the topical formulation is used for application to one or more regions of the skin, while the one or more regions do not comprise microbes excreting UV absorbing materials; and the topical formulation does not cause substantially skin irritation after being applied to the skin.

In still another embodiment, the topical formulation does not comprise an antimicrobial agent.

### Method of Use

Without intending to be limiting, but for purposes of exemplary embodiments, the compositions or formulations of the present invention take the form of a topical cream, ointment, gel, spray, foam, liquid, bar or lotion, be aerosolized, or be in powdered form. The compositions are formulated and used as creams, sticks, bars, wipes, serums, milks, pastes, aerosols, spray, foam or emulsions (e.g., water-in-oil, oil-in-water, silicone-in-water, water-in-oil-in-water, oil-in-water, oil-in-water-in-oil, water-in-silicone, oil-in-water-in-silicone, etc.), lotions, liquid solutions (e.g., aqueous or hydroalcoholic solutions), anhydrous bases (e.g., lipstick or powder), gels, ointments, eye jellies, solid forms, etc. for effective application, coverage and adhesion to the skin of a subject, for example, human skin.

In one embodiment of the present invention, the compositions or formulations described herein are applied as part of a user's routine makeup or personal care regimen.

In another embodiment of the present invention, the compositions or formulations described herein are used on an “as needed” basis.

In another embodiment of the present invention, the compositions described herein are formulated for topical application at least 1, 2, 3, 4, 5, 6, 7, or more times a day during use. In one preferred embodiment, the composition or formulation comprising one or more stains of microbes of the present invention is applied to skin every 40 to 90 minutes on a day with significant direct sun exposure. In another preferred embodiment, the composition or formulation of the present invention is used daily on days with incidental sun exposure. In still another preferred embodiment, the composition or formulation of the present invention comprising one or more stains of microbes are used daily or less when the microbes are demonstrated to have continued viability on the skin and continued production of UV absorbing materials.

### Examples

### Methods

The present invention includes formulations of microbial products, probiotics and combinations of each that allow restoration of the UV protective microbiome and/or its function.

Isolation of Microbes with UV Protective Properties

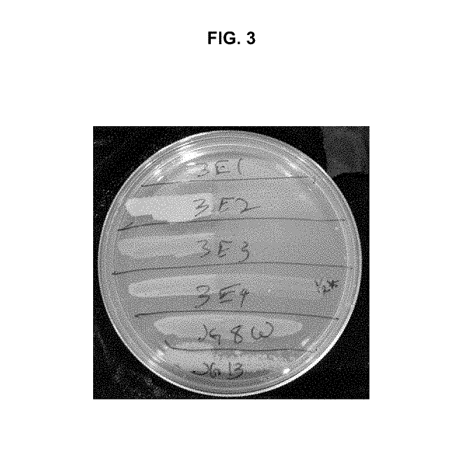

Using a variety of permissive culture plates (including but not limited to Mueller-Hinton, L B, and Lactobacilli MRS agars), microbes were cultured from the skin of individuals who had significant sun exposure (self reported) and did not regularly use sunscreen (self reported). The microbial colonies that grew on these plates were screened for their ability to resist UV radiation (FIG. 3). In this screen, microbes streaked to single colony isolate were subsequently spread across a culture plate. The plate was partially exposed to UV-B radiation (302 nm). The plate was then incubated in the dark until growth in the unexposed areas of the plate was observable. Growth in the UV-B irradiated part of the plate was observed for the UV resistant microbes. It is key to carry out this latter incubation in the dark to avoid selection for known light-mediated repair functions rather than the desired more direct protections against UV.

This technique was combined with replica plating techniques to allow for screening under a number of UV exposure conditions, including but not limited to a variety of time exposures (from 0 exposure to continuous exposure over the course of days) at 302 or 365 nm (applicable to any wavelength in the UVA, UVB, or UVC spectra).

### Characterizing the Production and Excretion of UV Absorbing Material

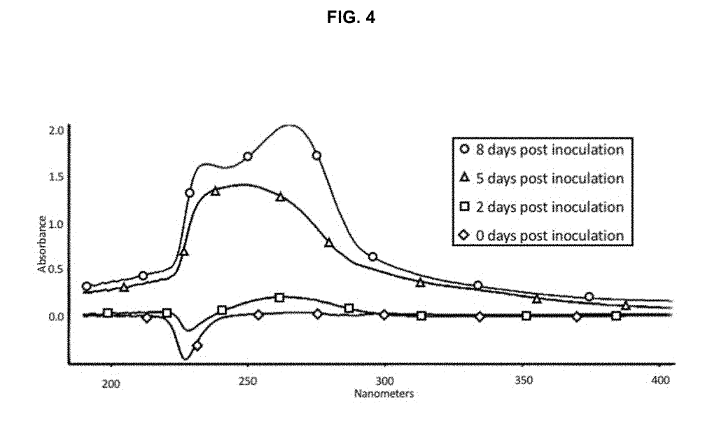

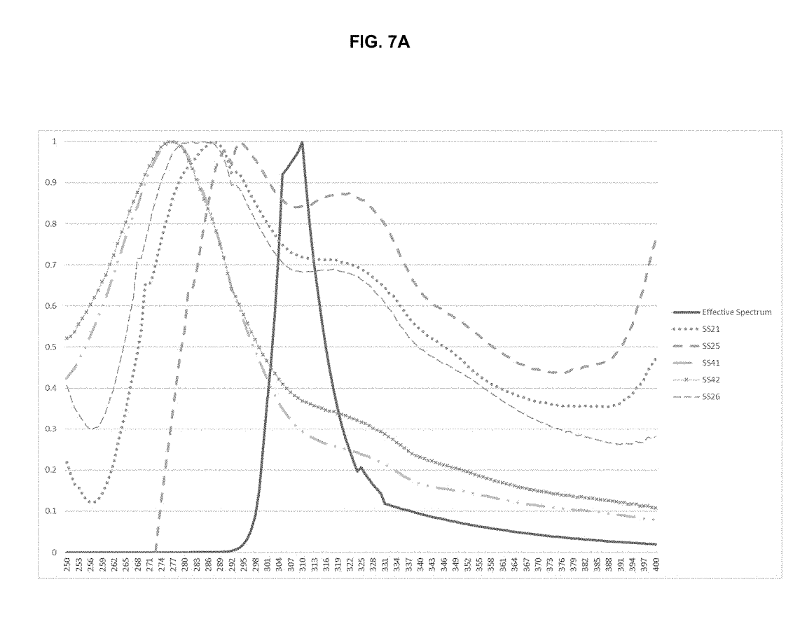

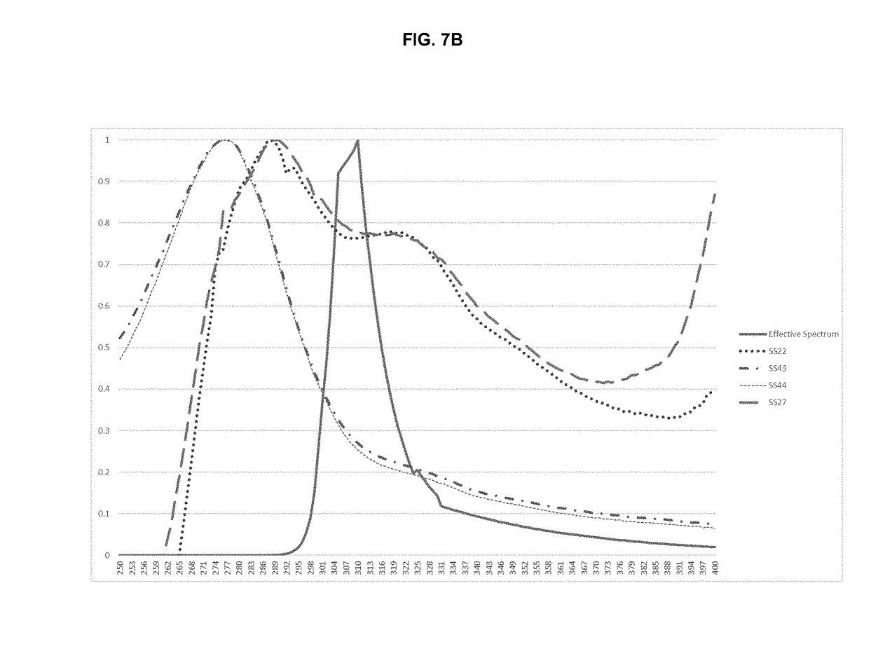

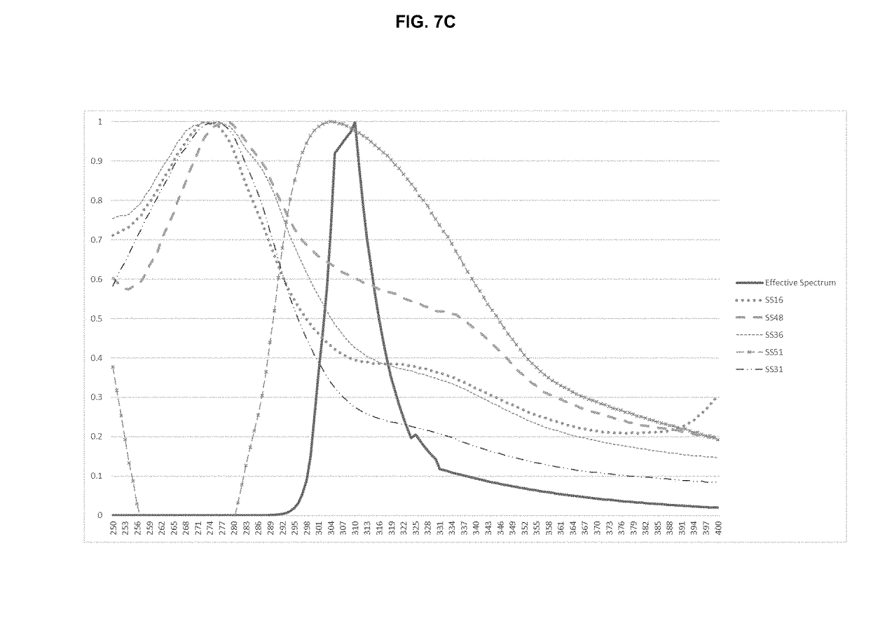

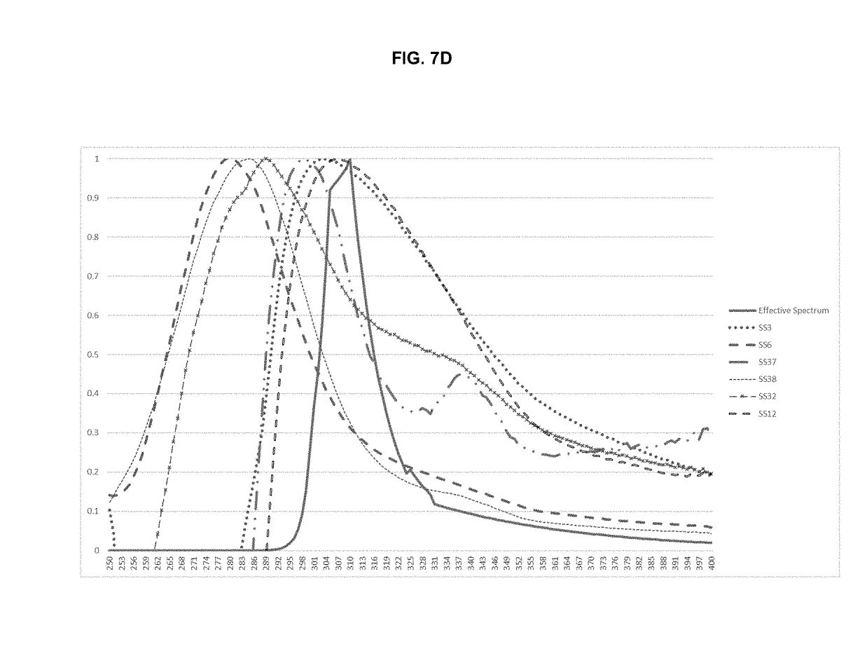

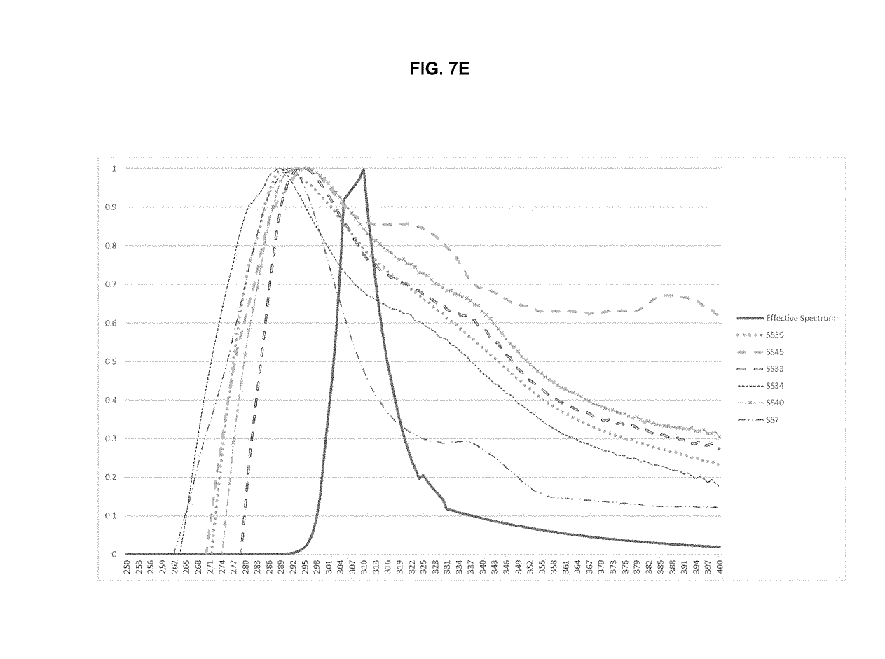

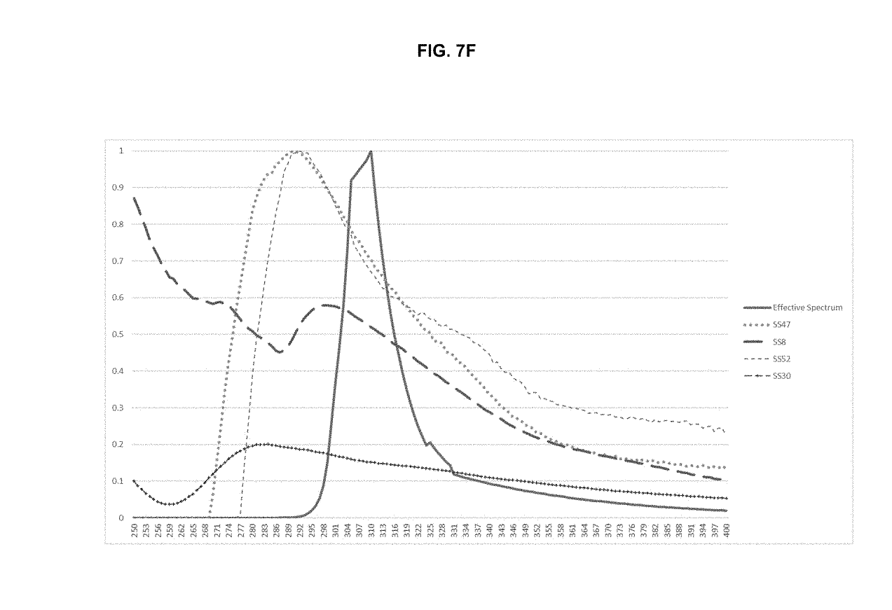

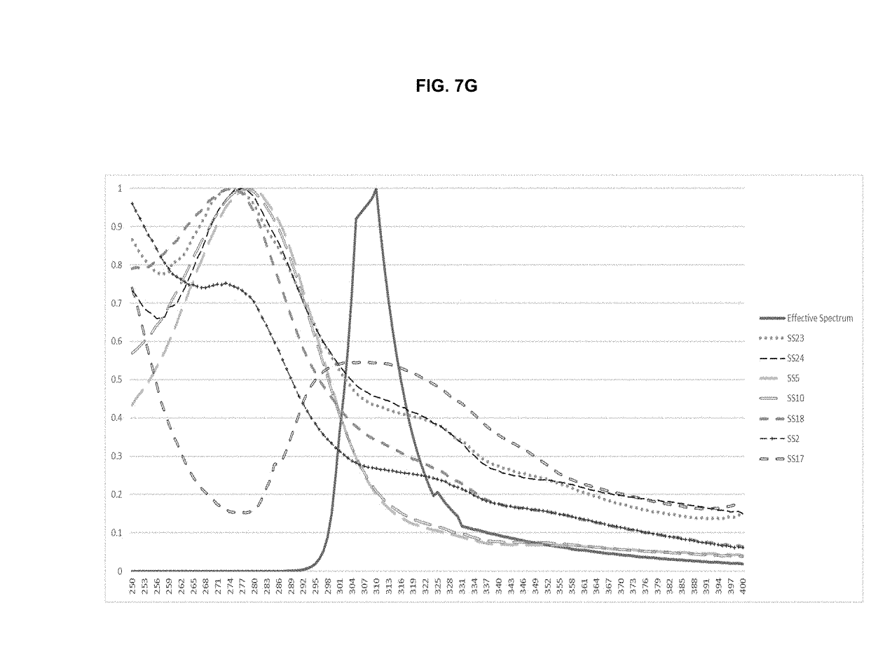

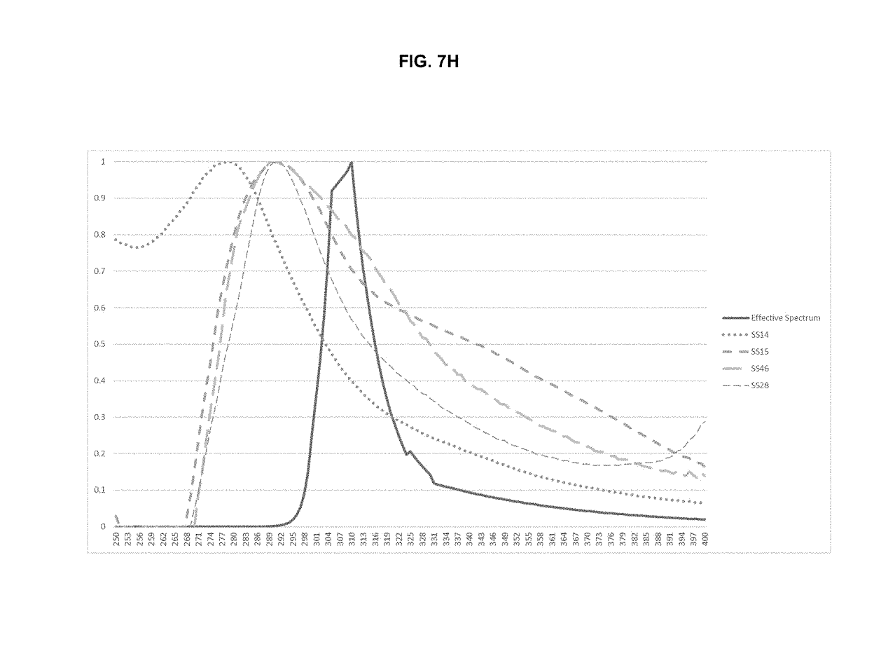

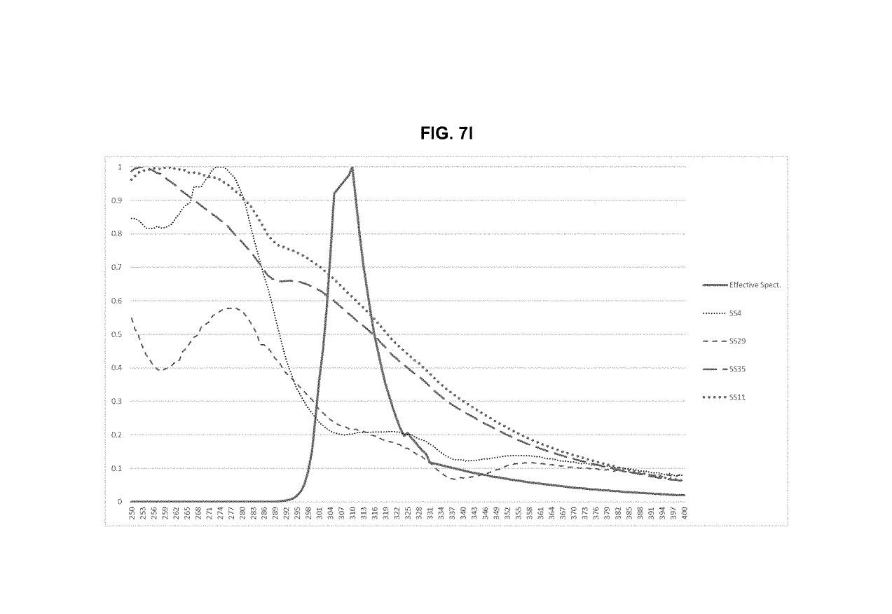

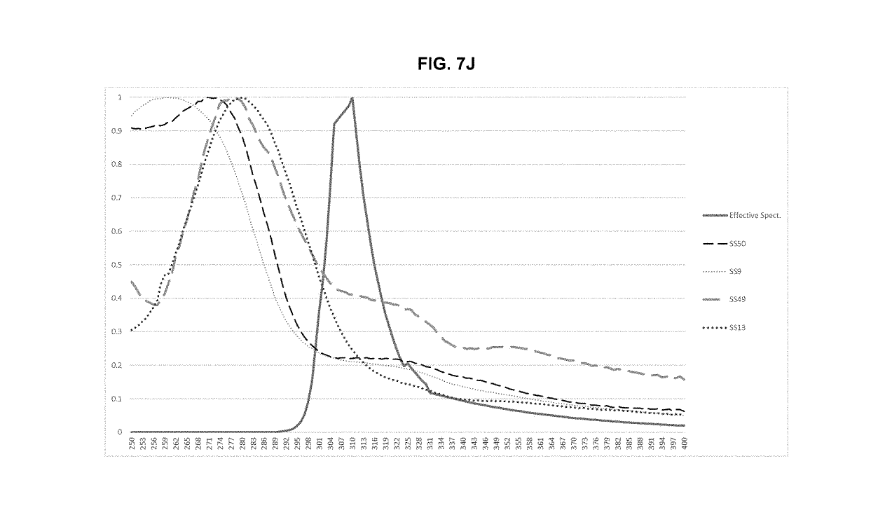

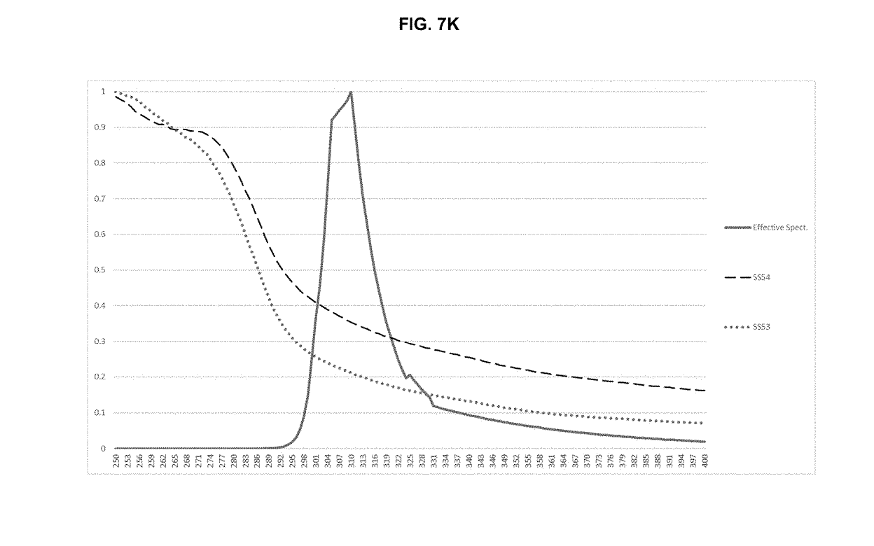

Those microbes that show resistance to UV radiation were further characterized for their ability to produce UV absorbing materials in liquid culture (FIG. 4 and FIG. 7). Microbes that have been streaked to clonal isolation were grown in any of a variety of media for varying periods of time. After the appropriate incubation period, the cultures were spun down to pellet the microbes. The supernatant was then assayed for UV absorbing materials using a UV-Vis spectrophotometer using media not inoculated with a microbe as the negative absorbance control.

### Assay for Reducing Photochemical By-products of UV Radiation

To assay for changes in photochemistry in the presence of microbes or the products of microbial growth, an indicator dye was added to solutions containing cultured media, microbes of interest, or control media. These solutions were then exposed to UV light and the changes in the rates of photochemistry were measured by the changes in conversion of the indicator dye in comparison to controls (control media or no UV exposure).

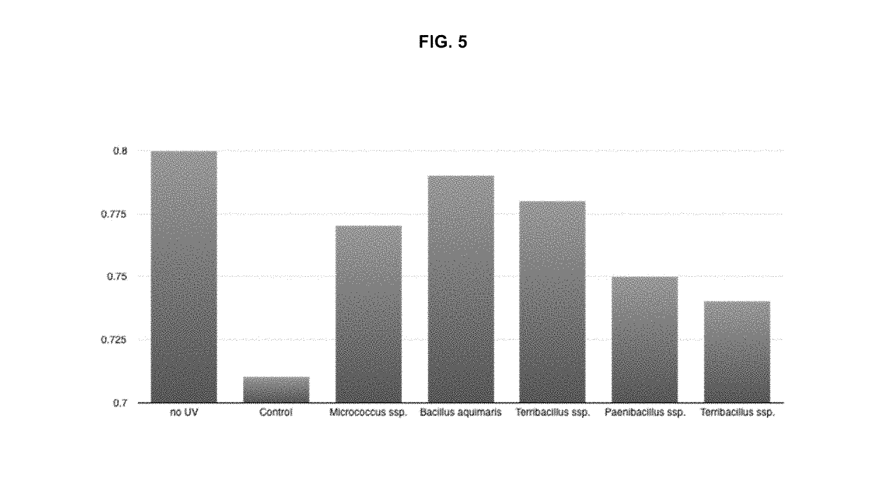

Fluorescent red dye (as shown in FIG. 5) or any easily measured indicator of photochemistry can be used in this assay. For fluorescent red, absorbance at 560 nm decreases with UV exposure, indicating the destruction of the dye. UV light at 302 nm was used in this case to induce photochemistry, but any UV wavelength is suitable. The presence of materials or inhibitors of photochemistry is shown by the reduction in the decrease in absorbance at 560 nm. Note that depending on the indicator used and the wavelength of radiation, the wavelength used to detect photochemical products will vary. 560 nm was chosen for these experiments as it represented a detectable signal.

### Characterization of Microbes Through 16S RNA Sequencing and Whole Genome Sequencing

Microbes were characterized to the genus and, if possible, to a species level through 16S RNA sequencing to screen out potential pathogens. It is important to note that 16S RNA identification, while giving a good indication of the relationship of the strain to others in the evolutionary tree, does not necessarily give an indication of all of the properties of the strain. As such, designations are indications of nearest evolutionary relative present in the searched database (National Center for Biotechnology Information (NCBO[Internet]. Bethesda (Md.): National Library of Medicine (US), National Center for Biotechnology Information; [1988]-[2017 Feb. 1]. Available from: www.ncbi.nlm.nih.gov) rather than a specific identification of a fully characterized strain. Similarly, the strains identified in this invention have specific properties that make them desirable for inclusion in this invention, but not all strains with a similar 16S RNA identification will have such properties.

Additional characterization, including determining the presence or absence of indicators of pathogenesis was done using whole genome sequencing for any bacteria that have close relatives considered to be pathogenic.

### Assay for Skin Irritation

Microbes from a pure plate culture were swabbed onto the forearm of a volunteer for these initial studies. Self-reported irritation and any change to the appearance of the skin was noted at 30 minutes and 10 hours post swab. Further characterization can be done using industry standard testing.

### Results

Methods have been developed that allow the selection and isolation of commensal microbial strains from human skin that secrete UV protective compounds, both directly through UV absorption and through the prevention or reduction of photochemical damage caused by UV radiation. These methods have been used to isolate and identify microbes that can be used to restore a natural UV protective function to the human skin microbiome.

In FIG. 3, an example set of microbes isolated from individuals with significant sun exposure who do not use sunscreen are shown to contain microbes that are resistant to exposure to 302 nm UV light, in stark contrast to species which do not excrete such compounds. Growth after UV exposures that kill non-resistant species was observed for species of Agrococcus, Bacillus, Microbacteriaceae, Micrococcus, Microbacterium, Paenibacillus, and Terribacillus. In stark contrast, all microbes from individuals who did not have significant sun exposure and use sunscreen showed no UV resistance (data not shown). FIG. 4 shows the UV absorbance spectrum of culture media after growth of one of these resistant bacteria, demonstrating the extracellular presence of the UV absorbing materials and confirming that the growth of these microbes on human skin would provide UV protective benefits to the host as well as to the microbe.

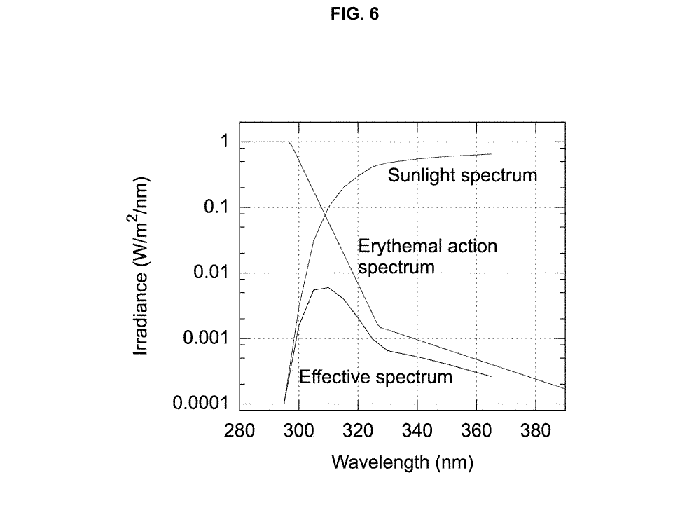

Using the methods described herein, many strains have been isolated that show desirable properties for inclusion in the formulation of the invention. Those showing significant absorbance in the area of the UV spectrum critical for sunburn (effective spectrum, see FIG. 6 for further explanation) have been further characterized. The absorbance spectra of media after growth of many of these microbes is shown in FIG. 7. While some of microbes secrete broad spectrum absorbent material, some are uniquely tailored, to the effective spectrum of sunlight to provide the commensal benefit of UV protection.

While direct absorption of UV radiation is the simplest mechanism of protecting the skin from UV rays, as described above, photochemistry due to UV rays is another known path to damage to the skin and which also plays a role in the increasing rates of skin cancer. FIG. 5 shows the reduction of photochemistry in the presence of several of the bacteria of interest and their products. Interestingly, a more substantial reduction in photochemistry was observed from the more UV resistant microbes, suggesting that this property is also useful for microbial resistance to UV radiation (strains shown in FIG. 5 are also identified in Table 1). The responsible factors in the form of cultured growth media or as secreted by microbes on the skin also reduce the exposure of the host to the potentially harmful breakdown products of chemicals applied to the skin either as sunscreen or for other purposes.

16S sequencing of the microbes is a key step in weeding out potential pathogens as such analysis can help to tell if a microbe is closely related to known pathogenic organisms. Table 2 describes identification to date of a subset of the microbes that are included in or used to produce products for the formulation of the invention based on both their efficacy in reducing the effects of UV rays on the skin and in their lack of pathogenesis. As discussed above, though 16S identification provides a good indication of the relationship of the strain to others in the evolutionary tree, it does not necessarily provide an indication of all of the properties of the strain to which it shows homology to. Likewise, not all strains sharing that identification with their close strains of this invention will have similar useful properties.

It has been suggested that bacteria to be considered for use as a probiotic should be susceptible to at least two clinically relevant antibiotics (Boriello S P, et al., Clin Infect Dis. 2003, 36(6):775-780). Antibiotic testing was done on the strains in consideration using standard techniques. Any with resistance to fewer than two of the antibiotics tested was removed from further consideration. See Table 2 for data on antibiotic resistance.

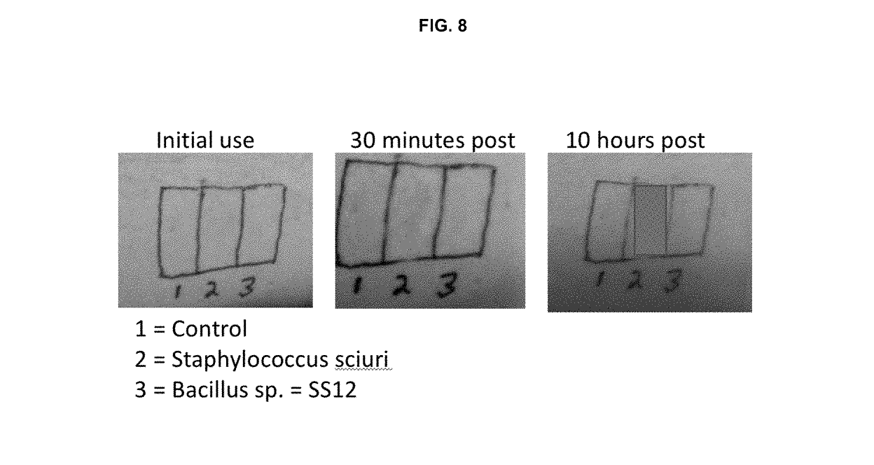

An important property for inclusion to the formulation of this invention is that the topical formulation does not cause substantial skin irritation when applied to the skin. Initial testing was done on a volunteer subject (see FIG. 8). Testing of two pilot strains showed irritation with one strain (Staphylococcus sciuri) and no irritation with a second (Bacillus sp.). All irritant strains are removed from further consideration.

This invention includes a way to restore a protective function of a microbiome that modern hygiene and antibiotic use have removed from the skin of many people. Given that the skin microbiome of humans in a pre-modern state provides a level of protection against UV rays, it is likely that the diversity of strains that can provide this protection is broad. A number of strains that excrete compounds absorbing in the important range of UV radiation for damage to the skin from individuals with significant sun exposure who use no sunscreen have been characterized in the Applicant's studies. We described herein some exemplary microbial species that may only touch the surface of the microbial diversity that fills this niche. Each new individual sampling produces additional strains potentially useful for inclusion or use in production of the formulation of the invention described herein.

While exemplary embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

TABLE 1
UV Resistance results of microbe strains.

| | UV resistance | |
|---|---|---|
| | − none | |
| Isolate | +++ extreme | Genus (16S) |
| H1E1 | ++ | Micrococcus ssp |
| H1O4Y | +++ | Micrococcus ssp. |
| H2E15 | +++ | Bacillus aquimaris |
| H2ER1R | ++ | Agrococcus or Microbacteriaceae |
| H3E4 | ++ | Microbacterium ssp. |
| JG13 | +++ | Terribacillus ssp. |
| JG2 | +++ | Paenibacillus ssp. |
| JG8W | +++ | Terribacillus ssp. |
| H2E9 | ++ | Bacillus ssp. |
| H2E11 | ++ | Bacillus ssp. |
| 3E1 | − | not identified |
| 3E2 | − | not identified |
| 3E3 | − | not identified |
| 3E4 | − | not identified |

TABLE 2
16S identification and antibiotic resistance characterization of
microbial strains. Identification by 16S is not an entirely unique identifier. That some of
these microbes are not uniquely identified suggests that these studies may be their first
characterization to this level. All strains considered show antibiotic susceptibility to 2 or
more clinically relevant antibiotics tested.

| CG ID | Nearest 16S Homology | Chloramphenicol | Erthromycin | Kanamycin |
|---|---|---|---|---|
| SS1 | Agrococcus or Micro-bacteriaceae | Susceptible | Intermediate | Resistant |
| SS2 | Agrococcus terreus | Susceptible | Susceptible | Intermediate |
| SS3 | Bacillus anthracis/cereus | Susceptible | Susceptible | Susceptible |
| SS4 | Bacillus aqulmaris | Susceptible | Susceptible | Susceptible |
| SS5 | Bacillus aqulmaris/marisflavi | Susceptible | Susceptible | Susceptible |
| SS6 | Bacillus firmus | Susceptible | Resistant | Susceptible |
| SS7 | Bacillus oleronius | Susceptible | Intermediate | Susceptible |
| SS8 | Bacillus simplexcBrevibacterium frigor | Susceptible | Susceptible | Susceptible |
| SS9 | Bacillus simplex/Brevibacterium frigor | Susceptible | Susceptible | Susceptible |
| SS10 | Bacillus sp. | Susceptible | Susceptible | Susceptible |
| SS11 | Bacillus sp. | Susceptible | Resistant | Susceptible |
| SS12 | Bacillus sp. BAB-4122 | Susceptible | Susceptible | Susceptible |
| SS13 | Bacillus ssp. | Susceptible | Susceptible | Susceptible |
| SS14 | Brevibacterium pityocampae | Susceptible | Susceptible | Intermediate |
| SS15 | Brevibacterium pityocampae | Susceptible | Susceptible | Intermediate |
| SS16 | Curtobacterium citreum | Susceptible | Susceptible | Intermediate |
| SS17 | Kocuria turfanensis | Susceptible | Susceptible | Susceptible |
| SS18 | Microbacterium lacticum/follorum | Susceptible | Susceptible | Resistant |
| SS19 | Microbacterium oleivorans/follorum | Susceptible | Susceptible | Susceptible |
| SS20 | Microbacterium ssp. | Susceptible | Susceptible | Resistant |
| SS21 | Micrococcus luteus | Susceptible | Susceptible | Susceptible |
| SS22 | Micrococcus sp. | Susceptible | Susceptible | Susceptible |
| SS23 | Micrococcus sp. | Susceptible | Susceptible | Resistant |
| SS24 | Micrococcus sp. | Susceptible | Susceptible | Susceptible |
| SS25 | Micrococcus yunnanensis | Susceptible | Susceptible | Susceptible |
| SS26 | Micrococcus yunnanensis | Susceptible | Susceptible | Susceptible |
| SS27 | Micrococcus yunnanensis | Susceptible | Susceptible | Susceptible |
| SS28 | Micrococcus yunnanensis | Susceptible | Susceptible | Susceptible |
| SS29 | Paenibacillus sp. | Susceptible | Susceptible | Intermediate |
| SS30 | Paracoccus acridae | Susceptible | Susceptible | Susceptible |
| SS31 | Staphylococcus arlettae | Susceptible | Resistant | Susceptible |
| SS32 | Staphylococcus capitis/caprae | Susceptible | Susceptible | Susceptible |
| SS33 | Staphylococcus epidermidis | Susceptible | Susceptible | Susceptible |
| SS34 | Staphylococcus epidermidis | Susceptible | Susceptible | Susceptible |
| SS35 | Staphylococcus epidermidis | Resistant | Resistant | Susceptible |
| SS36 | Staphylococcus haemolyticus | Susceptible | Susceptible | Resistant |
| SS37 | Staphylococcus haemolyticus | Susceptible | Susceptible | Resistant |
| SS38 | Staphylococcus haemolyticus | Susceptible | Susceptible | Susceptible |
| SS39 | Staphylococcus nepalensis | Resistant | Intermediate | Susceptible |
| SS40 | Staphylococcus nepalensis | Resistant | Intermediate | Susceptible |
| SS41 | Staphylococcus saprophytics | Susceptible | Intermediate | Susceptible |
| SS42 | Staphylococcus saprophytics | Susceptible | Susceptible | Susceptible |
| SS43 | Staphylococcus saprophyticus | Susceptible | Resistant | Susceptible |
| SS44 | Staphylococcus saprophyticus | Susceptible | Resistant | Susceptible |
| SS45 | Staphylococcus saprophyticus/xylosus | Susceptible | Susceptible | Susceptible |
| SS46 | Staphylococcus sp. | Susceptible | Susceptible | Susceptible |
| SS47 | Staphylococcus sp. | Susceptible | Susceptible | Resistant |
| SS48 | Terribacillus saccharophilus | Susceptible | Susceptible | Susceptible |
| SS49 | Terribacillus sp. | Susceptible | Susceptible | Intermediate |
| SS50 | Terribacillus sp. | Susceptible | Susceptible | Susceptible |
| SS51 | Bacillus thurigiensis/cereus | Susceptible | Susceptible | Susceptible |
| SS52 | Staphylococcus epidermidis | Susceptible | Susceptible | Susceptible |
| SS53 | Pseudomonas fulva | Resistant | Resistant | Susceptible |
| SS54 | Pseudomonas putida | Resistant | Resistant | Susceptible |

| | | | | | | Susceptible |
|---|---|---|---|---|---|---|
| CG ID | Neomycin | Novobiocin | Penicillin | Streptomycin | Tetracycline | Count |
| SS1 | Intermediate | Susceptible | Resistant | Susceptible | Susceptible | 4 |
| SS2 | Susceptible | Susceptible | Resistant | Susceptible | Susceptible | 6 |
| SS3 | Susceptible | Susceptible | Resistant | Susceptible | Susceptible | 7 |
| SS4 | Susceptible | Intermediate | Susceptible | Susceptible | Susceptible | 7 |
| SS5 | Susceptible | Susceptible | Susceptible | Susceptible | Susceptible | 8 |
| SS6 | Susceptible | Susceptible | Susceptible | Susceptible | Susceptible | 7 |
| SS7 | Susceptible | Susceptible | Susceptible | Susceptible | Susceptible | 7 |
| SS8 | Susceptible | Susceptible | Resistant | Susceptible | Susceptible | 7 |
| SS9 | Susceptible | Susceptible | Resistant | Susceptible | Susceptible | 7 |
| SS10 | Susceptible | Susceptible | Resistant | Susceptible | Susceptible | 7 |
| SS11 | Susceptible | Susceptible | Susceptible | Susceptible | Susceptible | 7 |
| SS12 | Susceptible | Susceptible | Susceptible | Susceptible | Susceptible | 8 |
| SS13 | Susceptible | Susceptible | Resistant | Susceptible | Susceptible | 7 |
| SS14 | Susceptible | Susceptible | Susceptible | Resistant | Susceptible | 6 |
| SS15 | Susceptible | Susceptible | Resistant | Resistant | Susceptible | 5 |
| SS16 | Susceptible | Susceptible | Resistant | Susceptible | Susceptible | 6 |
| SS17 | Susceptible | Susceptible | Susceptible | Susceptible | Susceptible | 8 |
| SS18 | Resistant | Susceptible | Susceptible | Susceptible | Susceptible | 6 |
| SS19 | Susceptible | Susceptible | Susceptible | Susceptible | Susceptible | 8 |
| SS20 | Susceptible | Susceptible | Resistant | Susceptible | Susceptible | 6 |
| SS21 | Susceptible | Susceptible | Resistant | Susceptible | Susceptible | 7 |
| SS22 | Susceptible | Susceptible | Susceptible | Resistant | Susceptible | 7 |
| SS23 | Intermediate | Susceptible | Resistant | Susceptible | Susceptible | 5 |
| SS24 | Susceptible | Susceptible | Susceptible | Susceptible | Susceptible | 8 |
| SS25 | Susceptible | Susceptible | Susceptible | Resistant | Susceptible | 7 |
| SS26 | Susceptible | Susceptible | Susceptible | Susceptible | Susceptible | 8 |
| SS27 | Susceptible | Susceptible | Susceptible | Resistant | Susceptible | 7 |
| SS28 | Susceptible | Susceptible | Susceptible | Susceptible | Susceptible | 8 |
| SS29 | Susceptible | Resistant | Resistant | Resistant | Susceptible | 4 |
| SS30 | Susceptible | Susceptible | Susceptible | Susceptible | Susceptible | 8 |
| SS31 | Susceptible | Susceptible | Susceptible | Susceptible | Susceptible | 7 |
| SS32 | Susceptible | Susceptible | Resistant | Susceptible | Susceptible | 7 |
| SS33 | Susceptible | Susceptible | Resistant | Susceptible | Susceptible | 7 |
| SS34 | Susceptible | Susceptible | Resistant | Susceptible | Susceptible | 7 |
| SS35 | Susceptible | Susceptible | Susceptible | Susceptible | Susceptible | 6 |
| SS36 | Resistant | Susceptible | Susceptible | Susceptible | Susceptible | 6 |
| SS37 | Intermediate | Susceptible | Susceptible | Susceptible | Susceptible | 6 |
| SS38 | Susceptible | Susceptible | Susceptible | Susceptible | Susceptible | 8 |
| SS39 | Susceptible | Susceptible | Susceptible | Susceptible | Susceptible | 6 |
| SS40 | Susceptible | Susceptible | Susceptible | Susceptible | Susceptible | 6 |
| SS41 | Susceptible | Susceptible | Susceptible | Susceptible | Susceptible | 7 |
| SS42 | Susceptible | Susceptible | Susceptible | Susceptible | Susceptible | 8 |
| SS43 | Susceptible | Susceptible | Susceptible | Susceptible | Susceptible | 7 |
| SS44 | Susceptible | Susceptible | Susceptible | Susceptible | Susceptible | 7 |
| SS45 | Susceptible | Susceptible | Susceptible | Susceptible | Susceptible | 8 |
| SS46 | Susceptible | Susceptible | Resistant | Susceptible | Susceptible | 7 |
| SS47 | Susceptible | Susceptible | Resistant | Susceptible | Susceptible | 6 |
| SS48 | Susceptible | Susceptible | Resistant | Susceptible | Susceptible | 7 |
| SS49 | Susceptible | Susceptible | Resistant | Resistant | Susceptible | 5 |
| SS50 | Susceptible | Susceptible | Susceptible | Susceptible | Susceptible | 8 |
| SS51 | Susceptible | Susceptible | Resistant | Susceptible | Susceptible | 7 |
| SS52 | Susceptible | Susceptible | Resistant | Susceptible | Susceptible | 7 |
| SS53 | Susceptible | Resistant | Resistant | Resistant | Resistant | 2 |
| SS54 | Susceptible | Resistant | Resistant | Resistant | Resistant | 2 |

### CITED REFERENCES

U.S. Pat. No. 6,613,755; Antimicrobial skin preparations containing organosilane quaternaries.
U.S. Pat. No. 6,114,377; Antimicrobial cosmetic compositions.
U.S. Pat. No. 8,193,244; Antimicrobial agents.
Publication number: US 2004/0228811A1; Sunscreen wipes having high sunscreen formulation transfer rate
Publication number: WO 2012046922; Novel use of antimicrobial peptides in regeneration of skin cells
Biniek, K., et al., Solar UV radiation reduces the barrier function of human skin, Proc. Natl. Acad. Sci. USA 2012; 109(42):17111-17116.
Boriello, S. P., et al., Safety of probiotics that contain lactobacilli or bifidobacteria, Clin. Infect. Dis. 2003; 36(6):775-780.
Bosch, R., et al., Mechanisms of photoaging and cutaneous photocarcinogenesis, and photoprotective strategies with phytochemicals, Antioxidants (Basel) 2015; 4(2): 248-268.
Brandt, L. J., Fecal transplantation for the treatment of clostridium difficile infection, Gastroenterol. Hepatol. (NY) 2012; 8(3):191-194.
Flament, F., et al., Effect of the sun on visible clinical signs of aging in Caucasian skin, Clin. Cosmet. Invest. Dermatol. 2013; 6: 221-232.
Gandini, S., et al., Meta-analysis of risk factors for cutaneous melanoma: II. Sun exposure, European Journal of Cancer 2005; 41(1):45-60.
Gonzalez, H., et al., Photostability of commercial sunscreens upon sun exposure and irradiation by ultraviolet lamps, BMC Dermatol. 2007; 7(1):1-9.
Grant, W. B., The effect of Solar UVB doses and vitamin D production, skin cancer action spectra, and smoking in explaining links between skin cancers and solid tumours, Eur. J. Cancer 2008; 44(1):12-15.
Guyuron, B., et al., Factors contributing to the facial aging of identical twins, Plastic & Reconstructive Surgery 2009; 123(4):1321-1331.
Hanson, K. M., et al., Sunscreen enhancement of UV-induced reactive oxygen species in the skin, Free Radical Biology & Medicine 2006; 41:1205-1212.
Keen, E. C., Paradigms of paradigms of pathogenesis: targeting the mobile genetic elements of disease, Frontiers in Cellular and Infection Microbiology 2012; 2 (article 161):1-3.
Klann, A., et al., Estrogen-like effects of ultraviolet screen 3-(4-methylbenzlidene)-camphor (Eusolex 6300) on cell proliferation and gene induction in mammalian and amphibian cells, Environ. Res. 2005; 97(3):274-281.
Knowland, J., et al., Sunlight-induced mutagenicity of a common sunscreen ingredient, FEBS Letters 1993; 324:309-313.
Krause, M., et al., Sunscreens: are they beneficial for health? An overview of endocrine disrupting properties of UV-filters, International Journal of Andrology 2012; 35(3):424-436.
Lloyd-Price, J., Strains, functions and dynamics in the expanded Human Microbiome Project, Nature 2017; 550:61-66.
McKinlay, A. F., et al., A reference action spectrum for ultraviolet induced erythema in human skin, CIE Journal 1987; 6(1):17-22.
Mosley, C. N., et al., Light-induced cytotoxicity and genotoxicity of a sunscreen agent, 2-phenylbenzimidazole in Salmonella typhimurium TA 102 and HaCaT keratinocytes, Int J. Environ. Res. Public Health 2007; 4(2):126-131.
National Center for Biotechnology Information (NCBI)[Internet]. Bethesda (Md.): National Library of Medicine (US), National Center for Biotechnology Information; [1988]-[2017 Feb. 1]. Available from: www.ncbi.nlm.nih.gov)
Pappas, A., Epidermal surface lipids, Dematoendocrinol. 2009; 1(2):72-76.
Sender, R., et al., Revised estimates for the number of human and bacteria cells in the body, PLoS Biol. 2016; 14(8):e1002533.
Stern, R. S., Clinical practice. Treatment of photoaging, N. Engl. J. Med. 2004; 350(15):1526-1534.

1. A topical formulation comprising one or more strains of microbes under appropriate conditions for continued bacterial viability and for application to the skin of a subject.

2. The topical formulation of claim 1, wherein the formulation is used for maintaining the appearance of the skin in a youthful phenotype or restoring the appearance of the skin to a youthful phenotype.

3. The topical formulation of claim 1, wherein the microbes excrete UV absorbing materials.

4. The topical formulation of claim 1, wherein the microbes excrete molecules that prevent or reduce photochemical damage caused by UV radiation.

5. The topical formulation of claim 1, wherein the one or more stains of microbes are selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp.

6. The topical formulation of claim 1, wherein the one or more stains of microbes are selected from the group consisting of Agrococcus terreus, Bacillus sp. BAB-4122, Bacillus anthracis, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus.

7. A topical formulation comprising products of bacterial growth for application to the skin of a subject, wherein the products of bacterial growth are selected from the group consisting of enzymes, metabolic by-products and peptides.

8. The topical formulation of claim 7, wherein the formulation is used for maintaining the appearance of the skin in a youthful phenotype or restoring the appearance of the skin to a youthful phenotype.

9. The topical formulation of claim 7, wherein the products of bacterial growth comprise UV absorbing materials.

10. The topical formulation of claim 7, wherein the products of bacterial growth prevent or reduce photochemical damage caused by UV radiation.

11. The topical formulation of claim 7, wherein the products of bacterial growth are from one or more stains of microbes selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp.

12. The topical formulation of claim 7, wherein the products of bacterial growth are from one or more stains of microbes selected from the group consisting of Agrococcus terreus, Bacillus sp. BAB-4122, Bacillus anthracis, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus.

13. A topical formulation comprising a combination of one or more strains of microbes under appropriate conditions for continued bacterial viability and products of bacterial growth for application to the skin, wherein the products of bacterial growth are selected from the group consisting of enzymes, metabolic by-products and peptides.

14. The topical formulation of claim 13, wherein the formulation is used for maintaining the appearance of the skin in a youthful phenotype or restoring the appearance of the skin to a youthful phenotype.

15. The topical formulation of claim 13, wherein the microbes excrete UV absorbing materials and the products of bacterial growth comprise UV absorbing materials.

16. The topical formulation of claim 13, wherein the microbes excrete UV absorbing materials or the products of bacterial growth comprise UV absorbing materials.

17. The topical formulation of claim 13, wherein the microbes produce materials that prevent or reduce photochemical damage caused by UV radiation and the products of bacterial growth prevent or reduce photochemical damage caused by UV radiation.

18. The topical formulation of claim 13, wherein the microbes produce materials that prevent or reduce photochemical damage caused by UV radiation or the products of bacterial growth prevent or reduce photochemical damage caused by UV radiation.

19. The topical formulation of claim 13, wherein the one or more stains of microbes are selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp; and/or
wherein the products of bacterial growth are from one or more stains of microbes selected from the group consisting of Agrococcus sp., Bacillus sp., Brevibacterium sp., Curtobacterium sp., Kocuria sp., Microbacterium sp., Micrococcus sp., Paenibacillus sp., Paracoccus sp., Phycibacter sp., Pseudomonas sp., Staphylococcus sp., and Terribacillus sp.

20. The topical formulation of claim 13, wherein the one or more stains of microbes are selected from the group consisting of Agrococcus terreus, Bacillus sp. BAB-4122, Bacillus anthracis, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus; and/or
wherein products of bacterial growth are from one or more stains of microbes selected from the group consisting of Agrococcus terreus, Bacillus sp. BAB-4122, Bacillus anthracis, Bacillus aquimaris, Bacillus cereus, Bacillus firmus, Bacillus marisflavi, Bacillus oleronius, Bacillus simplex, Bacillus thurigiensis, Brevibacterium frigoritolerans, Brevibacterium pityocampae, Curtobacterium citreum, Kocuria turfanensis, Microbacterium foliorum, Microbacterium lacticum, Microbacterium oleivorans, Micrococcus luteus, Micrococcus yunnanensis, Paracoccus acridae, Pseudomonas fulva, Pseudomonas putida, Staphylococcus arlettae, Staphylococcus capitis, Staphylococcus caprae, Staphylococcus epidermidis, Staphylococcus haemolyticus, Staphylococcus nepalensis, Staphylococcus saprophyticus, Staphylococcus xylosus, Terribacillus saccharophilus, Terribacillus aidingensis, and Terribacillus goriensus.

21. The topical formulation of claim 13, wherein the topical formulation does not comprise a synthetic small molecule UV absorption agent.

22. The topical formulation of claim 13, wherein the topical formulation is used for application to one or more regions of the skin wherein the one or more regions do not comprise microbes excreting UV absorbing materials.

23. The topical formulation of claim 13, wherein the topical formulation does not cause substantially skin irritation after being applied to the skin.

24. The topical formulation of claim 13, wherein the topical formulation does not comprise a synthetic small molecule UV absorption agent; and wherein the topical formulation is used for application to one or more regions of the skin wherein the one or more regions do not comprise microbes excreting UV absorbing materials.

25. The topical formulation of claim 13, wherein the topical formulation does not comprise a synthetic small molecule UV absorption agent; and wherein the topical formulation does not cause substantially skin irritation after being applied to the skin.

26. The topical formulation of claim 13, wherein the topical formulation is used for application to one or more regions of the skin wherein the one or more regions do not comprise microbes excreting UV absorbing materials; and wherein the topical formulation does not cause substantially skin irritation after being applied to the skin.

27. The topical formulation of claim 13, wherein the topical formulation does not comprise a synthetic small molecule UV absorption agent;
wherein the topical formulation is used for application to one or more regions of the skin wherein the one or more regions do not comprise microbes excreting UV absorbing materials; and
wherein the topical formulation does not cause substantially skin irritation after being applied to the skin.

28. The topical formulation of claim 13, wherein the topical formulation does not comprise an antimicrobial agent.
